(19) United States
(12) Patent Application Publication
(10) Pub. No.: US 2018/0207082 A1
(43) Pub. Date: Jul. 26, 2018

(54) HAIR COMPOSITION

(71) Applicant: Conopco, Inc., d/b/a UNILEVER, Englewood Cliffs, NJ (US)

(72) Inventor: Stephen Leonard BRIGGS, Bromborough, Wirral (GB)

(73) Assignee: Conopco, Inc., d/b/a UNILEVER, Englewood Cliffs, NJ (US)

(21) Appl. No.: 15/745,847

(22) PCT Filed: Jul. 1, 2016
(86) PCT No.: PCT/EP2016/065610

§ 371 Date: Jan. 18, 2018

(30) Foreign Application Priority Data
Jul. 24, 2015 (EP) 15178309.9

(51) Int. Cl. A61K 8/81; A61K 8/34; A61Q 5/12; A61Q 5/00

(52) U.S. Cl. CPC A61K 8/8152; A61K 8/345; A61K 2800/30; A61Q 5/002; A61K 2800/5426; A61Q 5/12

(57) ABSTRACT

The present invention provides: an aqueous hair care composition comprising; a. 0.5 to 3 w.t. of cationic polymer, b. one or more hair benefit agents, wherein the composition is substantially free of fatty materials, and wherein the benefit agent is selected from glycerine and silicone.

### FIELD OF THE INVENTION

The present invention relates to a hair composition with improved delivery of hair benefit agents to the hair.

### BACKGROUND OF THE INVENTION

Consumers desire efficacy from their hair care compositions. They are often disappointed by the amounts of hair benefit agents delivered to the hair fibres and by the amount of product which is removed from the hair at the end of a treatment. Low delivery of hair benefit agents to the hair fibre means that the desired benefit may not be achieved. The consumer is therefore often disappointed by hair care compositions.

Fatty materials are often used to structure hair care compositions. However, we have now found that use of cationic polymer to structure the composition rather than fatty materials provides a composition with significantly improved delivery to the hair. For example, significantly less product is left on a comb after treatment.

### SUMMARY OF THE INVENTION

Accordingly the present invention provides an aqueous hair care composition comprising;
a. 0.5 to 3 w.t. of cationic polymer
b. one or more hair benefit agents,
   wherein the composition is substantially free of fatty materials,
   and wherein the hair benefit agent is selected from glycerine and silicone.

### DETAILED DESCRIPTION OF THE INVENTION

Delivery to the hair fibre refers to delivery to the surface of the hair fibre and adsorption and/or absorption into the hair fibre.

### Fatty Material

The composition of the present invention is substantially free of fatty materials. By substantially free from fatty materials is meant that the composition comprises 0 to 5 w.t. % fatty material, preferably 0 to 3 w.t. % fatty material, more preferably 0 to 2% w.t. fatty material and most preferably 0 to 1 w.t. % fatty material.

Optionally the composition comprises 0.00001 to 5 w.t. % fatty material, preferably 0.00001 to 3 w.t. % fatty material, more preferably 0.00001 to 2% w.t. fatty material and most preferably 0.00001 to 1 w.t. % fatty material. The composition may comprise no fatty material at all.

The fatty materials are selected from fatty acids, fatty amides, fatty alcohols, fatty esters and mixtures thereof. Preferably the fatty materials are selected from fatty alcohols.

Preferably, the fatty materials comprises a fatty group having from 12 to 30 carbon atoms, more preferably 14 to 22.

Examples of suitable fatty alcohols that the composition is substantially free from include cetyl alcohol, stearyl alcohol and mixtures thereof.

### Cationic Polymer

The composition of the present invention comprises 0.5 to 3 w.t. % cationic polymer, preferably 0.5 to 2.5 w.t. %, more preferably, 0.75 to 2 w.t. % and most preferably 1 to 1.5 w.t. %.

By cationic polymer is meant a polymer with a net positive charge.

The polymers are preferably continuous phase thickening polymers and are preferably cross-linked.

The cationic polymer is preferably a synthetic cationic polymer. The synthetic polymer is preferably a Polyquaternium polymer.

In one embodiment the polyquaternium polymer is preferably a quarternarised poly dimethyl aminoethyl methacrylate (DMAEMA) polymer.

Commercially available suitable polymers are available such as Flocare ET 1037 from SNF, Ultragel 300 from BASF.

The cationic polymer preferably has a nitrogen content of more than 2 w.t. %, more preferably more than 3.5 w.t. %, most preferably more than 5 w.t. %. The nitrogen content is preferably less than 50 w.t. %.

The cationic polymer is preferably not a polysaccharide polymer.

The cationic polymer for use in the present invention preferably has a molecular weight of 0.75 to 4 million daltons, preferably 1 to 3 million daltons.

### Hair Benefit Agent

The composition of the present invention comprises a hair benefit agent selected from glycerine and silicone. These are materials which provide a benefit to the hair or scalp.

The glycerine is preferably present at a level of from 0.001 wt % to 50 wt %, more preferably 5 wt % to 50 wt %, even more preferably from 8 wt % to 30 wt %, most preferably from 10 wt % to 20 wt %, by weight of the total composition.

The silicone is preferably present at a level of from 0.001 wt % to 50 wt %, more preferably present in an amount of from 0.5 wt % to 10 wt %, most preferably from 1 wt % to 8 wt %, by weight of the total composition.

### Second Benefit Agent

The composition of the invention may further comprise a second benefit agent to provide a benefit to the hair or scalp.

The second benefit agent may be any material which provides a benefit to the hair or scalp. The one or more second benefit agents may preferably be selected from conditioning agents, antimicrobial compounds, colouring agents, styling agents, UV-absorbing compounds, fluorescers, hair strengthening agents (such as fibre repair agents or fibre rebuilding agents), antioxidants, perfumes, proteins, straightening agents and mixtures thereof.

The second benefit agent is not intended to be glycerine or the same silicone as the hair benefit agent.

In one embodiment the second benefit agent may comprise a protein.

Examples of suitable proteins are collagen, elastin, keratin, casein, wheat protein, soya protein silk and vegetable proteins.

The composition may preferably comprise up to 50 w.t. % of the second benefit agent e.g. 0.001 to 50 w.t. % second benefit agent. Suitable levels of second benefit agents will depend on the second benefit agent and the desired result. For example it may be preferable to have only 0.1 w.t. % of perfume in the composition, but 2 w.t. % protein.

### Water

The composition of the present invention is an aqueous composition. By aqueous composition is meant that the composition comprises water.

A percentage of the water present in the composition may be considered as a hair benefit agent, because hydration levels in hair fibres can be regarded as important for hair health.

### Cationic Quaternary Surfactant

The composition of the present invention preferably comprises a cationic quaternary surfactant.

Preferably, the cationic quaternary surfactants have the formula N+R¹R²R³R⁴ wherein R¹, R², R³ and R⁴ are independently (C₁ to C₃₀) alkyl or benzyl. Preferably, one, two or three of R¹, R², R³ and R⁴ are independently (C₄ to C₃₀) alkyl and the other R¹, R², R³ and R⁴ group or groups are (C₁-C₆) alkyl or benzyl. More preferably, one or two of R¹, R², R³ and R⁴ are independently (C₆ to C₃₀) alkyl and the other R¹, R², R³ and R⁴ groups are (C₁-C₆) alkyl or benzyl groups. Optionally, the alkyl groups may comprise one or more ester (—OCO— or —COO—) and/or ether (—O—) linkages within the alkyl chain. Alkyl groups may optionally be substituted with one or more hydroxyl groups. Alkyl groups may be straight chain or branched and, for alkyl groups having 3 or more carbon atoms, cyclic. The alkyl groups may be saturated or may contain one or more carbon-carbon double bonds (eg, oleyl). Alkyl groups are optionally ethoxylated on the alkyl chain with one or more ethyleneoxy groups.

Suitable cationic quarternary surfactants for use in conditioner compositions according to the invention include Stearamidopropyldimethylamine, cetyltrimethylammonium chloride, behenyltrimethylammonium chloride, cetylpyridinium chloride, tetramethylammonium chloride, tetraethylammonium chloride, octyltrimethylammonium chloride, dodecyltrimethylammonium chloride, hexadecyltrimethylammonium chloride, octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, dihydrogenated tallow dimethyl ammonium chloride (eg, Arquad 2HT/75 from Akzo Nobel), cocotrimethylammonium chloride, PEG-2-oleammonium chloride and the corresponding hydroxides thereof. Further suitable cationic surfactants include those materials having the CTFA designations Quaternium-5, Quaternium-31 and Quaternium-18. Mixtures of any of the foregoing materials may also be suitable. A particularly useful cationic surfactant for use according to the present invention is behenyltrimethylammonium chloride, available commercially, for example as GENAMIN BTLF from Aako.

The composition may preferably comprise 0.001 w.t. % to 2.5 w.t. % cationic quaternary surfactant, more preferably 0.01 w.t. % to 1.5 w.t. %, most preferably 0.05 w.t. % to 0.75 w.t. %.

### Product Form

The composition of the present invention may be a leave on or a rinse off composition. The composition may be in the form of a conditioner, mask, mousse, serum, cream, lotion or styling product. Preferably the composition is in the form of a conditioner or hair mask.

Preferably the composition of the current invention does not comprise an anionic cleansing surfactant.

### Other Components

The composition of the present invention may comprise other components suitable for hair care compositions, for example; viscosity modifiers, solvents, preservatives, fragrances, colorants, pH adjusters, emollients.

### Use

The present invention provides:

Use of an aqueous hair care composition comprising;
a. 0.5 to 3 w.t. of cationic polymer,
b. one or more hair benefit agents,

wherein the composition is substantially free of fatty materials,

and wherein the benefit agent is selected from glycerine and silicone, to deliver the one or more hair benefit agents to a hair fibre.

### Method

The present invention also provides a method of delivering a hair benefit agent to a hair fibre by use of a hair care composition of the present invention, wherein the method comprises applying the composition to the hair.

The method preferably comprises the steps of;
a. Applying to the composition to the hair
b. Massaging the composition into the hair
c. Removing the composition from the hair

Removal of the composition may preferably be achieved by combing, brushing, washing, rinsing or rubbing.

The composition may optionally be left on the hair for a period of up to 90 minutes, with a minimum period of 1 second (i.e. immediate rinsing), before removal.

### Example

TABLE 1
Inventive composition 1

| Ingredient | Trade name and supplier | w.t. % |
|---|---|---|
| Polyquaternium 37 | | 2 |
| Cetearyl Alcohol | | 0.5 |
| Behentrimonium Chloride | GENAMIN BTLF | 0.306 (70% |
| and Dipropylene Glycol | from Aako | active) |
| Glycerin | | 10 |
| Cyclopentasiloxane | | 5 |
| Dimethacone | DC2-7134 from | 0.71 |
| | Dow Corning | |
| Polyurethane-39(20% active) | Luvigel Star | 0.5 |
| | from BASF | |
| Disodium ETDA | | 0.5 |
| Minors | | 1.06 |
| Water | | To 100 |

TABLE 2
Comparative composition A

| Ingredient | | w.t. % |
|---|---|---|
| Cetearyl Alcohol | | 6 |
| Behentrimonium Chloride | GENAMIN BTLF, | 1.5 (70% |
| and Dipropylene Glycol | Aako | active) |
| glycerine | | 1 |
| Dimethacone | DC 5-7134 from | 4.3 |
| | Dow Corning | |
| Polyurethane-39(20% active) | Luvigel Star | 0.5 |
| | from BASF | |
| Disodium ETA | | 0.5 |
| Stearamidopropyl Dimethylamine | | 1.5 |
| Lactic acid | | 1.2 (88% |
| | | active) |
| Petroleum | | 0.3 |
| Mineral oil | | 0.2 |
| Minors | | 1.95 |
| Water | | To 100 |

### Method

1 g of the composition was applied along the length of a hair switch.
The composition was massaged into the hair for 30 seconds.
The hair switch was combed through the full length of the switch, once.
The amount of composition removed by the comb was weighed.

TABLE 3
Results

| | 1 | A |
|---|---|---|
| Weight of composition removed | 0.18 g | 0.64 g |
| % of composition delivered to the hair fibre. | 82% | 36% |

The example clearly shows that a significant amount more of the inventive composition was taken up by the hair switch.

1-12. (canceled)

13. An aqueous hair care composition comprising:
a) 0.5 to 3 wt. % of cationic polymer and
b) one or more hair benefit agents,
wherein: the composition is substantially free of fatty materials; the cationic polymer is a Polyquaternium polymer; and the benefit agent is glycerine, which is present at a level of from 10 wt. % to 50 wt. % of the hair care composition.

14. The composition according to claim 13 which further comprises a second benefit agent selected from conditioning agents, antimicrobial compounds, colouring agents, styling agents, UV-absorbing compounds, fluorescers, hair strengthening agents, antioxidants, perfumes, and mixtures thereof.

15. The composition according to claim 13 wherein the format of the hair care composition is a conditioner or a rinse off hair mask.

16. The composition according to claim 13 wherein glycerine is present at a level of from 10 wt % to 30 wt % of the hair care composition.

17. The composition according to claim 13 wherein glycerine is present at a level of from 10 wt % to 20 wt % of the hair care composition.

18. A method of delivering a hair benefit agent to hair fibre, the method comprising applying the composition of claim 13 to the hair fibre.
